(19) United States
(12) Patent Application Publication
(10) Pub. No.: US 2025/0345140 A1
(43) Pub. Date: Nov. 13, 2025

(54) STRUGGLING MOTION IDENTIFICATION AND DETECTION FOR FLEXIBLE ELONGATE DEVICES

(71) Applicant: Intuitive Surgical Operations, Inc., Sunnyvale, CA (US)

(72) Inventors: Shibing Liu, San Jose, CA (US); Samuel B. Schorr, Sunnyvale, CA (US)

(73) Assignee: Intuitive Surgical Operations, Inc., Sunnyvale, CA (US)

(21) Appl. No.: 19/204,145

(22) Filed: May 9, 2025

Related U.S. Application Data
(60) Provisional application No. 63/644,786, filed on May 9, 2024.

(51) Int. Cl. A61B 34/00; A61B 34/30; G16H 40/63

(52) U.S. Cl. CPC A61B 34/74; A61B 34/30; G16H 40/63; A61B 2034/301

(57) ABSTRACT

A medical system includes a manipulator assembly and a control system. The manipulator assembly includes actuators that control an articulation state of a flexible elongate device by changing tensions applied to pull wires of the flexible elongate device. The control system controls the actuators to change the articulation state of the flexible elongate device using a control scheme that defines an upper-bounded tension applied to each of the pull wires. The control system further determines that a commanded articulation state of the flexible elongate device cannot be achieved by control of a first actuator without the first actuator applying more than the upper-bounded tension to a first pull wire. The control system also controls a second actuator to change an amount of tension applied to a second pull wire to achieve the commanded articulation state without the first actuator applying more than the upper-bounded tension to the first pull wire.

### CROSS-REFERENCE TO RELATED APPLICATIONS

This application claims the benefit of priority under 35 U S.C. § 119 (e) to U.S. Provisional Patent Application Ser. No. 63/644,786, filed on May 9, 2024, which is hereby incorporated by reference herein in its entirety.

### FIELD

Disclosed embodiments relate to improved robotic and/or medical (including surgical) devices, systems, and methods.

### BACKGROUND

Minimally invasive medical techniques are intended to reduce the amount of tissue that is damaged during medical procedures, thereby reducing patient recovery time, discomfort, and harmful side effects. Such minimally invasive techniques may be performed through natural orifices in a patient anatomy or through one or more surgical incisions. Through these natural orifices or incisions, physicians may insert minimally invasive medical instruments (including surgical, diagnostic, therapeutic, and/or biopsy instruments) to reach a target tissue location. One such minimally invasive technique is to use a flexible and/or steerable elongate device, such as a flexible catheter or bronchoscope, which can be inserted into anatomic passageways and navigated toward a region of interest within the patient's anatomy.

However, various elongate devices may experience some difficulty in reaching a desired region of interest. For example, an articulation of the flexible elongate device may be impaired by friction. When the friction becomes too severe, the desired articulation may become unachievable for the elongate device.

### SUMMARY

The following presents a simplified summary of various examples described herein and is not intended to identify key or critical elements or to delineate the scope of the claims.

In some examples, embodiments of the disclosure relate to a medical system, comprising: a manipulator assembly comprising a plurality of actuators configured to control an articulation state of a flexible elongate device based on changing a plurality of tensions applied to a plurality of pull wires of the flexible elongate device; and a control system coupled to the manipulator assembly, the control system configured to: control the plurality of actuators to change the articulation state of the flexible elongate device using a control scheme, the control scheme defining an upper-bounded tension applied to each of the plurality of pull wires; determine that a commanded articulation state of the flexible elongate device cannot be achieved by control of a first actuator of the plurality of actuators without the first actuator applying more than the upper-bounded tension to a first pull wire of the plurality of pull wires; and control a second actuator of the plurality of actuators to change an amount of tension applied to a second pull wire of the plurality of pull wires to achieve the commanded articulation state without the first actuator applying more than the upper-bounded tension to the first pull wire.

In some examples, embodiments of the disclosure relate to a non-transitory machine-readable medium comprising a plurality of machine-readable instructions executed by one or more processors associated with a medical system, the medical system comprising a manipulator assembly comprising a plurality of actuators configured to control an articulation state of a flexible elongate device based on changing a plurality of tensions applied to a plurality of pull wires of the flexible elongate device, the plurality of machine-readable instructions causing the one or more processors to perform a method comprising: controlling the plurality of actuators to change the articulation state of the flexible elongate device using a control scheme, the control scheme defining an upper-bounded tension applied to each of the plurality of pull wires; determining that a commanded articulation state of the flexible elongate device cannot be achieved by control of a first actuator of the plurality of actuators without the first actuator applying more than the upper-bounded tension to a first pull wire of the plurality of pull wires; and controlling a second actuator of the plurality of actuators to change an amount of tension applied to a second pull wire of the plurality of pull wires to achieve the commanded articulation state without the first actuator applying more than the upper-bounded tension to the first pull wire.

In some examples, embodiments of the disclosure relate to a method for operating a medical system, the medical system comprising a manipulator assembly comprising a plurality of actuators configured to control an articulation state of a flexible elongate device based on changing a plurality of tensions applied to a plurality of pull wires of the flexible elongate device, the method comprising: controlling the plurality of actuators to change the articulation state of the flexible elongate device using a control scheme, the control scheme defining an upper-bounded tension applied to each of the plurality of pull wires; determining that a commanded articulation state of the flexible elongate device cannot be achieved by control of a first actuator of the plurality of actuators without the first actuator applying more than the upper-bounded tension to a first pull wire of the plurality of pull wires; and controlling a second actuator of the plurality of actuators to change an amount of tension applied to a second pull wire of the plurality of pull wires to achieve the commanded articulation state without the first actuator applying more than the upper-bounded tension to the first pull wire.

It is to be understood that both the foregoing general description and the following detailed description are illustrative and explanatory in nature and are intended to provide an understanding of the present disclosure without limiting the scope of the present disclosure. In that regard, additional aspects, features, and advantages of the present disclosure will be apparent to one skilled in the art from the following detailed description.

### BRIEF DESCRIPTIONS OF THE DRAWINGS

FIG. 1 is a simplified diagram of a medical system according to some embodiments.

FIG. 2A is a simplified diagram of a medical instrument system according to some embodiments.

FIG. 2B is a simplified diagram of a medical instrument including a medical tool within an elongate device according to some embodiments.

FIGS. 3A and 3B are simplified diagrams of side views of a patient coordinate space including a medical instrument mounted on an insertion assembly according to some embodiments.

FIG. 4 is a flowchart of methods according to some embodiments.

FIG. 5A is an illustration of a scenario in which an actual articulation state of a flexible elongate device is different from a commanded articulation state.

FIG. 5B is an illustration of an actual articulation state of a flexible elongate device vs a commanded articulation state of the flexible elongate device according to some embodiments.

FIGS. 5C and 5D are illustrations of a direct articulation trajectory and a non-direct articulation trajectory based on detected friction according to some embodiments.

FIGS. 5E and 5F are illustrations of a direct articulation trajectory and a non-direct articulation trajectory based on detected friction according to some embodiments.

FIG. 5G are illustrations of actuator torque or force limits according to some embodiments.

Embodiments of the present disclosure and their advantages are best understood by referring to the detailed description that follows. It should be appreciated that like reference numerals are used to identify like elements illustrated in one or more of the figures, wherein showings therein are for purposes of illustrating embodiments of the present disclosure and not for purposes of limiting the same.

### DETAILED DESCRIPTION

In the following description, specific details are set forth describing some embodiments consistent with the present disclosure. Numerous specific details are set forth in order to provide a thorough understanding of the embodiments. It will be apparent, however, to one skilled in the art that some embodiments may be practiced without some or all of these specific details. The specific embodiments disclosed herein are meant to be illustrative but not limiting. One skilled in the art may realize other elements that, although not specifically described here, are within the scope and the spirit of this disclosure. In addition, to avoid unnecessary repetition, one or more features shown and described in association with one embodiment may be incorporated into other embodiments unless specifically described otherwise or if the one or more features would make an embodiment non-functional. In some instances, well known methods, procedures, components, and circuits have not been described in detail so as not to unnecessarily obscure aspects of the embodiments.

This disclosure describes various instruments and portions of instruments in terms of their state in three-dimensional space. As used herein, the term “position” refers to the location of an object or a portion of an object in a three-dimensional space (e.g., three degrees of translational freedom along Cartesian x-, y-, and z-coordinates). As used herein, the term “orientation” refers to the rotational placement of an object or a portion of an object (e.g., one or more degrees of rotational freedom such as, roll, pitch, and yaw). As used herein, the term “pose” refers to the position of an object or a portion of an object in at least one degree of translational freedom and to the orientation of that object or portion of the object in at least one degree of rotational freedom (e.g., up to six total degrees of freedom). As used herein, the term “shape” refers to a set of poses, positions, and/or orientations measured along an object. As used herein, the term “distal” refers to a position that is closer to a procedural site and the term “proximal” refers to a position that is further from the procedural site. Accordingly, the distal portion or distal end of an instrument is closer to a procedural site than a proximal portion or proximal end of the instrument when the instrument is being used as designed to perform a procedure.

Embodiments of the disclosure include medical systems and methods for operating such medical systems. A medical system (such as medical systems that use flexible elongate devices (e.g., catheters or endoscopes) may include a medical instrument that can be driven along one or more articulation degrees of freedom.

Driving along the articulation degrees of freedom may involve articulation of an articulable body portion of the flexible elongate device, such as a distal portion of the flexible elongate device. In one example, the articulation degrees of freedom include articulation across pitch and yaw axes. Driving along the articulation degrees of freedom may be performed to navigate the distal end of the flexible elongate device toward a target tissue and/or to orient an end effector towards target tissue to perform a medical operation such as a biopsy, an ablation, an electroporation, etc. The articulation of the articulable body portion may be driven from a proximal portion of the elongate device, e.g., using pull wires, driven by actuators. The pull wires may be actively put under mechanical tension by the actuators to cause the articulation.

The tension on the pull wires may be limited. For example, the tension may be limited to an upper bound tension (also referred to as a maximum tension) that is set based on one or more factors such as a strength of the pull wire, the strength of other structures that are exposed to resulting forces and/or torques, a maximum output (force or torque) of an actuator, software-specified limits, effect of tension on structural integrity or life of the pull wire, etc.

The pull wires may further encounter friction that reduces the amount of force available to articulate the articulable body portion. The presence of friction may be non-uniform, for example, when a particular section of a pull wire is more worn than other sections of the pull wire, or when pull wires are more bent resulting from the curvature(s) of the flexible elongate. When such a worn and/or highly bent section is in contact with a guiding element (e.g., a coil pipe) surrounding the pull wire, friction may be increased, in comparison to when a non-worn or straighter section is in contact with the guiding element.

The friction as described, as well as motion against tissue, may result in struggling motion of the flexible elongate device. An example of such struggling motion includes an incomplete execution of a commanded or desired articulation of the articulable body portion. An incomplete execution of a command may involve a portion of a commanded articulation having been completed, but not the entire command. An incomplete execution may also involve a complete failure to perform the command. This may result in the flexible elongate device being less curved than as commanded or desired by the user.

In some embodiments, the struggling motion of the flexible elongate device is detected and resolved. For example, incomplete execution of a commanded articulation is detected, and actions are taken to enable completion of the commanded articulation.

A more detailed discussion of the medical system, the medical instrument operations, and methods addressing an incomplete execution of a desired commanded motion (e.g., articulation), including resulting benefits, is provided below in reference to the figures.

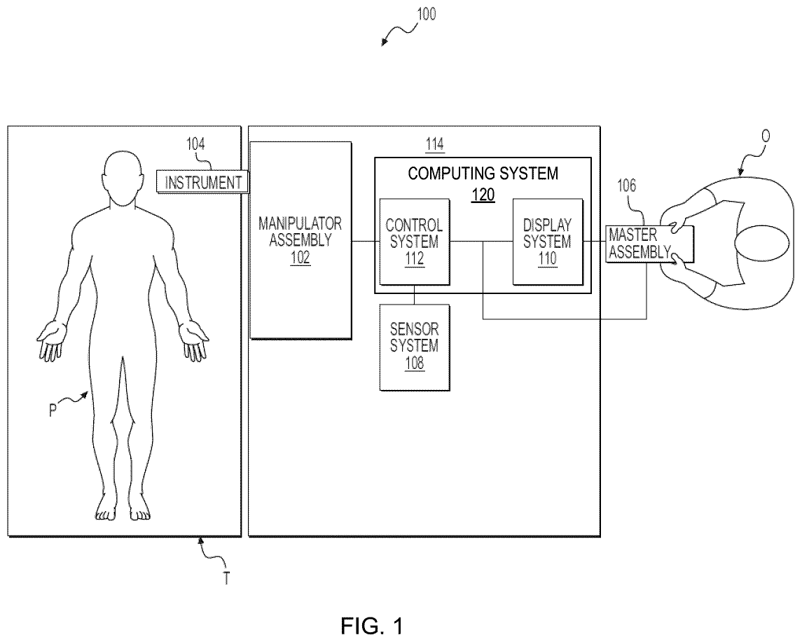

Turning to the figures, FIG. 1 is a simplified diagram of a medical system 100 according to some embodiments. The medical system 100 may be suitable for use in, for example, surgical, diagnostic (e.g., biopsy), or therapeutic (e.g., ablation, electroporation, etc.) procedures. While some embodiments are provided herein with respect to such procedures, any reference to medical or surgical instruments and medical or surgical methods is non-limiting. The systems, instruments, and methods described herein may be used for animals, human cadavers, animal cadavers, portions of human or animal anatomy, non-surgical diagnosis, as well as for industrial systems, general or special purpose robotic systems, general or special purpose teleoperational systems, or robotic medical systems.

As shown in FIG. 1, medical system 100 may include a manipulator assembly 102 that controls the operation of a medical instrument 104 in performing various procedures on a patient P. Medical instrument 104 may extend into an internal site within the body of patient P via an opening in the body of patient P. The manipulator assembly 102 may be teleoperated, non-teleoperated, or a hybrid teleoperated and non-teleoperated assembly with one or more degrees of freedom of motion that may be motorized and/or one or more degrees of freedom of motion that may be non-motorized (e.g., manually operated). The manipulator assembly 102 may be mounted to and/or positioned near a patient table T. A master assembly 106 allows an operator O (e.g., a surgeon, a clinician, a physician, or other user) to control the manipulator assembly 102. In some examples, the master assembly 106 allows the operator O to view the procedural site or other graphical or informational displays. In some examples, the manipulator assembly 102 may be excluded from the medical system 100 and the instrument 104 may be controlled directly by the operator O. In some examples, the manipulator assembly 102 may be manually controlled by the operator O. Direct operator control may include various handles and operator interfaces for hand-held operation of the instrument 104.

The master assembly 106 may be located at a surgeon's console which is in proximity to (e.g., in the same room as) a patient table T on which patient P is located, such as at the side of the patient table T. In some examples, the master assembly 106 is remote from the patient table T, such as in in a different room or a different building from the patient table T. The master assembly 106 may include one or more control devices for controlling the manipulator assembly 102. The control devices may include any number of a variety of input devices, such as joysticks, trackballs, scroll wheels, directional pads, buttons, data gloves, trigger-guns, hand-operated controllers, voice recognition devices, motion or presence sensors, and/or the like.

The manipulator assembly 102 supports the medical instrument 104 and may include a kinematic structure of links that provide a set-up structure. The links may include one or more non-servo-controlled links (e.g., one or more links that may be manually positioned and locked in place) and/or one or more servo-controlled links (e.g., one or more links that may be controlled in response to commands, such as from a control system 112). The manipulator assembly 102 may include a plurality of actuators (e.g., motors) that drive inputs on the medical instrument 104 in response to commands, such as from the control system 112. The actuators may include drive systems that move the medical instrument 104 in various ways when coupled to the medical instrument 104. For example, one or more actuators may advance medical instrument 104 into a naturally or surgically created anatomic orifice. Actuators may control articulation of the medical instrument 104, such as by moving the distal end (or any other portion) of medical instrument 104 in multiple degrees of freedom. These degrees of freedom may include three degrees of linear motion (e.g., linear motion along the X, Y, Z Cartesian axes) and in three degrees of rotational motion (e.g., rotation about the X, Y, Z Cartesian axes). One or more actuators may control rotation of the medical instrument about a longitudinal axis. Actuators can also be used to move an articulable end effector of medical instrument 104, such as for grasping tissue in the jaws of a biopsy device and/or the like, or may be used to move or otherwise control tools (e.g., imaging tools, ablation tools, biopsy tools, electroporation tools, etc.) that are inserted within the medical instrument 104.

The medical system 100 may include a sensor system 108 with one or more sub-systems for receiving information about the manipulator assembly 102 and/or the medical instrument 104. Such sub-systems may include a position sensor system (e.g., that uses electromagnetic (EM) sensors or other types of sensors that detect position or location); a shape sensor system for determining the position, orientation, speed, velocity, pose, and/or shape of a distal end and/or of one or more segments along a flexible body of the medical instrument 104; a visualization system (e.g., using a color imaging device, an infrared imaging device, an ultrasound imaging device, an x-ray imaging device, a fluoroscopic imaging device, a computed tomography (CT) imaging device, a magnetic resonance imaging (MRI) imaging device, or some other type of imaging device) for capturing images, such as from the distal end of medical instrument 104 or from some other location; and/or actuator position sensors such as resolvers, encoders, potentiometers, and the like that describe the rotation and/or orientation of the actuators controlling the medical instrument 104.

The medical system 100 may include a display system 110 for displaying an image or representation of the procedural site and the medical instrument 104. Display system 110 and master assembly 106 may be oriented so physician O can control medical instrument 104 and master assembly 106 with the perception of telepresence.

In some embodiments, the medical instrument 104 may include a visualization system, which may include an image capture assembly that records a concurrent or real-time image of a procedural site and provides the image to the operator O through one or more displays of display system 110. The image capture assembly may include various types of imaging devices. The concurrent image may be, for example, a two-dimensional image or a three-dimensional image captured by an endoscope positioned within the anatomical procedural site. In some examples, the visualization system may include endoscopic components that may be integrally or removably coupled to medical instrument 104. Additionally or alternatively, a separate endoscope, attached to a separate manipulator assembly, may be used with medical instrument 104 to image the procedural site. The visualization system may be implemented as hardware, firmware, software or a combination thereof which interact with or are otherwise executed by one or more computer processors, such as of the control system 112.

Display system 110 may also display an image of the procedural site and medical instruments, which may be captured by the visualization system. In some examples, the medical system 100 provides a perception of telepresence to the operator O. For example, images captured by an imaging device at a distal portion of the medical instrument 104 may be presented by the display system 110 to provide the perception of being at the distal portion of the medical instrument 104 to the operator O. The input to the master assembly 106 provided by the operator O may move the distal portion of the medical instrument 104 in a manner that corresponds with the nature of the input (e.g., distal tip turns right when a trackball is rolled to the right) and results in corresponding change to the perspective of the images captured by the imaging device at the distal portion of the medical instrument 104. As such, the perception of telepresence for the operator O is maintained as the medical instrument 104 is moved using the master assembly 106. The operator O can manipulate the medical instrument 104 and hand controls of the master assembly 106 as if viewing the workspace in substantially true presence, simulating the experience of an operator that is physically manipulating the medical instrument 104 from within the patient anatomy.

In some examples, the display system 110 may present virtual images of a procedural site that are created using image data recorded pre-operatively (e.g., prior to the procedure performed by the medical instrument system 200) or intra-operatively (e.g., concurrent with the procedure performed by the medical instrument system 200), such as image data created using computed tomography (CT), magnetic resonance imaging (MRI), positron emission tomography (PET), fluoroscopy, thermography, ultrasound, optical coherence tomography (OCT), thermal imaging, impedance imaging, laser imaging, nanotube X-ray imaging, and/or the like. The virtual images may include two-dimensional, three-dimensional, or higher-dimensional (e.g., including, for example, time based or velocity-based information) images. In some examples, one or more models are created from pre-operative or intra-operative image data sets and the virtual images are generated using the one or more models.

In some examples, for purposes of imaged guided medical procedures, display system 110 may display a virtual image that is generated based on tracking the location of medical instrument 104. For example, the tracked location of the medical instrument 104 may be registered (e.g., dynamically referenced) with the model generated using the pre-operative or intra-operative images, with different portions of the model correspond with different locations of the patient anatomy. As the medical instrument 104 moves through the patient anatomy, the registration is used to determine portions of the model corresponding with the location and/or perspective of the medical instrument 104 and virtual images are generated using the determined portions of the model. This may be done to present the operator O with virtual images of the internal procedural site from viewpoints of medical instrument 104 that correspond with the tracked locations of the medical instrument 104.

The medical system 100 may also include the control system 112, which may include processing circuitry that implements the some or all of the methods or functionality discussed herein. The control system 112 may include at least one memory and at least one processor for controlling the operations of the manipulator assembly 102, the medical instrument 104, the master assembly 106, the sensor system 108, and/or the display system 110. Control system 112 may include instructions (e.g., a non-transitory machine-readable medium storing the instructions) that when executed by the at least one processor, configures the one or more processors to implement some or all of the methods or functionality discussed herein. While the control system 112 is shown as a single block in FIG. 1, the control system 112 may include two or more separate data processing circuits with one portion of the processing being performed at the manipulator assembly 102, another portion of the processing being performed at the master assembly 106, and/or the like. In some examples, the control system 112 may include other types of processing circuitry, such as application-specific integrated circuits (ASICs) and/or field-programmable gate array (FPGAs). The control system 112 may be implemented using hardware, firmware, software, or a combination thereof.

In some examples, the control system 112 may receive feedback from the medical instrument 104, such as force and/or torque feedback. Responsive to the feedback, the control system 112 may transmit signals to the master assembly 106. In some examples, the control system 112 may transmit signals instructing one or more actuators of the manipulator assembly 102 to move the medical instrument 104. In some examples, the control system 112 may transmit informational displays regarding the feedback to the display system 110 for presentation or perform other types of actions based on the feedback.

The control system 112 may include a virtual visualization system to provide navigation assistance to operator O when controlling the medical instrument 104 during an image-guided medical procedure. Virtual navigation using the virtual visualization system may be based upon an acquired pre-operative or intra-operative dataset of anatomic passageways of the patient P. The control system 112 or a separate computing device may convert the recorded images, using programmed instructions alone or in combination with operator inputs, into a model of the patient anatomy. The model may include a segmented two-dimensional or three-dimensional composite representation of a partial or an entire anatomic organ or anatomic region. An image data set may be associated with the composite representation. The virtual visualization system may obtain sensor data from the sensor system 108 that is used to compute an (e.g., approximate) location of the medical instrument 104 with respect to the anatomy of patient P. The sensor system 108 may be used to register and display the medical instrument 104 together with the pre-operatively or intra-operatively recorded images. For example, PCT Publication WO 2016/191298 (published Dec. 1, 2016 and titled “Systems and Methods of Registration for Image Guided Surgery”), which is incorporated by reference herein in its entirety, discloses example systems.

During a virtual navigation procedure, the sensor system 108 may be used to compute the (e.g., approximate) location of the medical instrument 104 with respect to the anatomy of patient P. The location can be used to produce both macro-level (e.g., external) tracking images of the anatomy of patient P and virtual internal images of the anatomy of patient P. The system may include one or more electromagnetic (EM) sensors, fiber optic sensors, and/or other sensors to register and display a medical instrument together with pre-operatively recorded medical images. For example, U.S. Pat. No. 8,900,131 (filed May 13, 2011 and titled “Medical System Providing Dynamic Registration of a Model of an Anatomic Structure for Image-Guided Surgery”), which is incorporated by reference herein in its entirety, discloses example systems.

Medical system 100 may further include operations and support systems (not shown) such as illumination systems, steering control systems, irrigation systems, and/or suction systems. In some embodiments, the medical system 100 may include more than one manipulator assembly and/or more than one master assembly. The exact number of manipulator assemblies may depend on the medical procedure and space constraints within the procedural room, among other factors. Multiple master assemblies may be co-located or they may be positioned in separate locations. Multiple master assemblies may allow more than one operator to control one or more manipulator assemblies in various combinations.

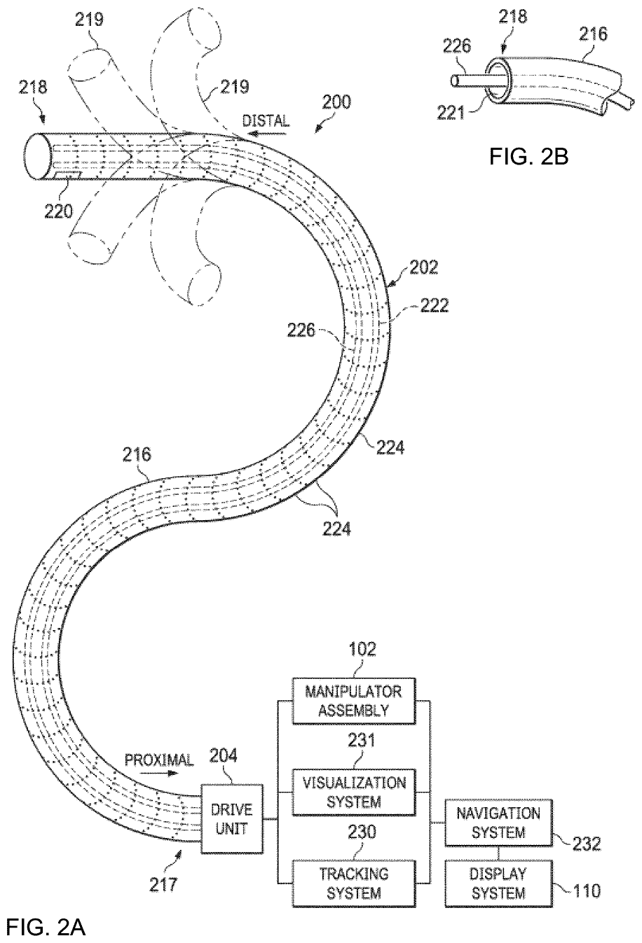

FIG. 2A is a simplified diagram of a medical instrument system 200 according to some embodiments. The medical instrument system 200 includes a flexible elongate device 202 (also referred to as elongate device 202), a drive unit 204, and a medical tool 226 that collectively is an example of a medical instrument 104 of a medical system 100. The medical system 100 may be a teleoperated system, a non-teleoperated system, or a hybrid teleoperated and non-teleoperated system, as explained with reference to FIG. 1. A visualization system 231, tracking system 230, and navigation system 232 are also shown in FIG. 2A and are example components of the control system 112 of the medical system 100. In some examples, the medical instrument system 200 may be used for non-teleoperational exploratory procedures or in procedures involving traditional manually operated medical instruments, such as endoscopy. The medical instrument system 200 may be used to gather (e.g., measure) a set of data points corresponding to locations within anatomic passageways of a patient, such as patient P.

The elongate device 202 is coupled to the drive unit 204. The elongate device 202 includes a channel 221 through which the medical tool 226 may be inserted. The elongate device 202 navigates within patient anatomy to deliver the medical tool 226 to a procedural site. The elongate device 202 includes a flexible body 216 having a proximal end 217 and a distal end 218. In some examples, the flexible body 216 may have an approximately 3 mm outer diameter. Other flexible body outer diameters may be larger or smaller.

Medical instrument system 200 may include the tracking system 230 for determining the position, orientation, speed, velocity, pose, and/or shape of the flexible body 216 at the distal end 218 and/or of one or more segments 224 along flexible body 216, as will be described in further detail below. The tracking system 230 may include one or more sensors and/or imaging devices. The flexible body 216, such as the length between the distal end 218 and the proximal end 217, may include multiple segments 224. The tracking system 230 may be implemented using hardware, firmware, software, or a combination thereof. In some examples, the tracking system 230 is part of control system 112 shown in FIG. 1.

Tracking system 230 may track the distal end 218 and/or one or more of the segments 224 of the flexible body 216 using a shape sensor 222. The shape sensor 222 may include an optical fiber aligned with the flexible body 216 (e.g., provided within an interior channel of the flexibly body 216 or mounted externally along the flexible body 216). In some examples, the optical fiber may have a diameter of approximately 200 μm. In other examples, the diameter may be larger or smaller. The optical fiber of the shape sensor 222 may form a fiber optic bend sensor for determining the shape of flexible body 216. Optical fibers including Fiber Bragg Gratings (FBGs) may be used to provide strain measurements in structures in one or more dimensions. Various systems and methods for monitoring the shape and relative position of an optical fiber in three dimensions, which may be applicable in some embodiments, are described in U.S. Patent Application Publication No. 2006/0013523 (filed Jul. 13, 2005 and titled “Fiber optic position and shape sensing device and method relating thereto”); U.S. Pat. No. 7,772,541 (filed on Mar. 12, 2008 and titled “Fiber Optic Position and/or Shape Sensing Based on Rayleigh Scatter”); and U.S. Pat. No. 8,773,650 (filed on Sep. 2, 2010 and titled “Optical Position and/or Shape Sensing”), which are all incorporated by reference herein in their entireties. Sensors in some embodiments may employ other suitable strain sensing techniques, such as Rayleigh scattering, Raman scattering, Brillouin scattering, and Fluorescence scattering.

In some examples, the shape of the flexible body 216 may be determined using other techniques. For example, a history of the position and/or pose of the distal end 218 of the flexible body 216 can be used to reconstruct the shape of flexible body 216 over an interval of time (e.g., as the flexible body 216 is advanced or retracted within a patient anatomy). In some examples, the tracking system 230 may alternatively and/or additionally track the distal end 218 of the flexible body 216 using a position sensor system 220. Position sensor system 220 may be a component of an EM sensor system with the position sensor system 220 including one or more position sensors. Although the position sensor system 220 is shown as being near the distal end 218 of the flexible body 216 to track the distal end 218, the number and location of the position sensors of the position sensor system 220 may vary to track different regions along the flexible body 216. In one example, the position sensors include conductive coils that may be subjected to an externally generated electromagnetic field. Each coil of position sensor system 220 may produce an induced electrical signal having characteristics that depend on the position and orientation of the coil relative to the externally generated electromagnetic field. The position sensor system 220 may measure one or more position coordinates and/or one or more orientation angles associated with one or more portions of flexible body 216. In some examples, the position sensor system 220 may be configured and positioned to measure six degrees of freedom, e.g., three position coordinates X, Y, Z and three orientation angles indicating pitch, yaw, and roll of a base point. In some examples, the position sensor system 220 may be configured and positioned to measure five degrees of freedom, e.g., three position coordinates X, Y, Z and two orientation angles indicating pitch and yaw of a base point. Further description of a position sensor system, which may be applicable in some embodiments, is provided in U.S. Pat. No. 6,380,732 (filed Aug. 11, 1999 and titled “Six-Degree of Freedom Tracking System Having a Passive Transponder on the Object Being Tracked”), which is incorporated by reference herein in its entirety.

In some embodiments, the tracking system 230 may alternately and/or additionally rely on a collection of pose, position, and/or orientation data stored for a point of an elongate device 202 and/or medical tool 226 captured during one or more cycles of alternating motion, such as breathing. This stored data may be used to develop shape information about the flexible body 216. In some examples, a series of position sensors (not shown), such as EM sensors like the sensors in position sensor 220 or some other type of position sensors may be positioned along the flexible body 216 and used for shape sensing. In some examples, a history of data from one or more of these position sensors taken during a procedure may be used to represent the shape of elongate device 202, particularly if an anatomic passageway is generally static.

FIG. 2B is a simplified diagram of the medical tool 226 within the elongate device 202 according to some embodiments. The flexible body 216 of the elongate device 202 may include the channel 221 sized and shaped to receive the medical tool 226. In some embodiments, the medical tool 226 may be used for procedures such as diagnostics, imaging, surgery, biopsy, ablation, illumination, irrigation, suction, electroporation, etc. Medical tool 226 can be deployed through channel 221 of flexible body 216 and operated at a procedural site within the anatomy. Medical instrument 226 may be, for example, an image capture probe, a biopsy tool (e.g., a needle, grasper, brush, etc.), an ablation tool (e.g., a laser ablation tool, radio frequency (RF) ablation tool, cryoablation tool, thermal ablation tool, heated liquid ablation tool, etc.), an electroporation tool, and/or another surgical, diagnostic, or therapeutic tool. In some examples, the medical tool 226 may include an end effector having a single working member such as a scalpel, a blunt blade, an optical fiber, an electrode, and/or the like. Other end types of end effectors may include, for example, forceps, graspers, scissors, staplers, clip appliers, and/or the like. Other end effectors may further include electrically activated end effectors such as electrosurgical electrodes, transducers, sensors, and/or the like.

The medical tool 226 may be a biopsy tool used to remove sample tissue or a sampling of cells from a target anatomic location. In some examples, the biopsy tool is a flexible needle. The biopsy tool may further include a sheath that can surround the flexible needle to protect the needle and interior surface of the channel 221 when the biopsy tool is within the channel 221. The medical tool 226 may be an image capture probe that includes a distal portion with a stereoscopic or monoscopic camera that may be placed at or near the distal end 218 of flexible body 216 for capturing images (e.g., still or video images). The captured images may be processed by the visualization system 231 for display and/or provided to the tracking system 230 to support tracking of the distal end 218 of the flexible body 216 and/or one or more of the segments 224 of the flexible body 216. The image capture probe may include a cable for transmitting the captured image data that is coupled to an imaging device at the distal portion of the image capture probe. In some examples, the image capture probe may include a fiber-optic bundle, such as a fiberscope, that couples to a more proximal imaging device of the visualization system 231. The image capture probe may be single-spectral or multi-spectral, for example, capturing image data in one or more of the visible, near-infrared, infrared, and/or ultraviolet spectrums. The image capture probe may also include one or more light emitters that provide illumination to facilitate image capture. In some examples, the image capture probe may use ultrasound, x-ray, fluoroscopy, CT, MRI, or other types of imaging technology.

In some examples, the image capture probe is inserted within the flexible body 216 of the elongate device 202 to facilitate visual navigation of the elongate device 202 to a procedural site and then is replaced within the flexible body 216 with another type of medical tool 226 that performs the procedure. In some examples, the image capture probe may be within the flexible body 216 of the elongate device 202 along with another type of medical tool 226 to facilitate simultaneous image capture and tissue intervention, such as within the same channel 221 or in separate channels. A medical tool 226 may be advanced from the opening of the channel 221 to perform the procedure (or some other functionality) and then retracted back into the channel 221 when the procedure is complete. The medical tool 226 may be removed from the proximal end 217 of the flexible body 216 or from another optional instrument port (not shown) along flexible body 216.

In some examples, the elongate device 202 may include integrated imaging capability rather than utilize a removable image capture probe. For example, the imaging device (or fiber-optic bundle) and the light emitters may be located at the distal end 218 of the elongate device 202. The flexible body 215 may include one or more dedicated channels that carry the cable(s) and/or optical fiber(s) between the distal end 218 and the visualization system 231. Here, the medical instrument system 200 can perform simultaneous imaging and tool operations.

In some examples, the medical tool 226 is capable of controllable articulation. The medical tool 226 may house cables (which may also be referred to as pull wires), linkages, or other actuation controls (not shown) that extend between its proximal and distal ends to controllably bend the distal end of medical tool 226, such as discussed herein for the flexible elongate device 202. The medical tool 226 may be coupled to a drive unit 204 and the manipulator assembly 102. In these examples, the elongate device 202 may be excluded from the medical instrument system 200 or may be a flexible device that does not have controllable articulation. Steerable instruments or tools, applicable in some embodiments, are further described in detail in U.S. Pat. No. 7,316,681 (filed on Oct. 4, 2005 and titled “Articulated Surgical Instrument for Performing Minimally Invasive Surgery with Enhanced Dexterity and Sensitivity”) and U.S. Pat. No. 9,259,274 (filed Sep. 30, 2008 and titled “Passive Preload and Capstan Drive for Surgical Instruments”), which are incorporated by reference herein in their entireties.

The flexible body 216 of the elongate device 202 may also or alternatively house cables, linkages, or other steering controls (not shown) that extend between the drive unit 204 and the distal end 218 to controllably bend the distal end 218 as shown, for example, by broken dashed line depictions 219 of the distal end 218 in FIG. 2A. In some examples, at least four cables are used to provide independent up-down steering to control a pitch of the distal end 218 and left-right steering to control a yaw of the distal end 281. In these examples, the flexible elongate device 202 may be a steerable catheter. Examples of steerable catheters, applicable in some embodiments, are described in detail in PCT Publication WO 2019/018736 (published Jan. 24, 2019 and titled “Flexible Elongate Device Systems and Methods”), which is incorporated by reference herein in its entirety.

In embodiments where the elongate device 202 and/or medical tool 226 are actuated by a teleoperational assembly (e.g., the manipulator assembly 102), the drive unit 204 may include drive inputs that removably couple to and receive power from drive elements, such as actuators, of the teleoperational assembly. In some examples, the elongate device 202 and/or medical tool 226 may include gripping features, manual actuators, or other components for manually controlling the motion of the elongate device 202 and/or medical tool 226. The elongate device 202 may be steerable or, alternatively, the elongate device 202 may be non-steerable with no integrated mechanism for operator control of the bending of distal end 218. In some examples, one or more channels 221 (which may also be referred to as lumens), through which medical tools 226 can be deployed and used at a target anatomical location, may be defined by the interior walls of the flexible body 216 of the elongate device 202.

In some examples, the medical instrument system 200 (e.g., the elongate device 202 or medical tool 226) may include a flexible bronchial instrument, such as a bronchoscope or bronchial catheter, for use in examination, diagnosis, biopsy, and/or treatment of a lung. The medical instrument system 200 may also be suited for navigation and treatment of other tissues, via natural or surgically created connected passageways, in any of a variety of anatomic systems, including the colon, the intestines, the kidneys and kidney calices, the brain, the heart, the circulatory system including vasculature, and/or the like.

The information from the tracking system 230 may be sent to the navigation system 232, where the information may be combined with information from the visualization system 231 and/or pre-operatively obtained models to provide the physician, clinician, surgeon, or other operator with real-time position information. In some examples, the real-time position information may be displayed on the display system 110 for use in the control of the medical instrument system 200. In some examples, the navigation system 232 may utilize the position information as feedback for positioning medical instrument system 200. Various systems for using fiber optic sensors to register and display a surgical instrument with surgical images, applicable in some embodiments, are provided in U.S. Pat. No. 8,900,131 (filed May 13, 2011 and titled “Medical System Providing Dynamic Registration of a Model of an Anatomic Structure for Image-Guided Surgery”), which is incorporated by reference herein in its entirety.

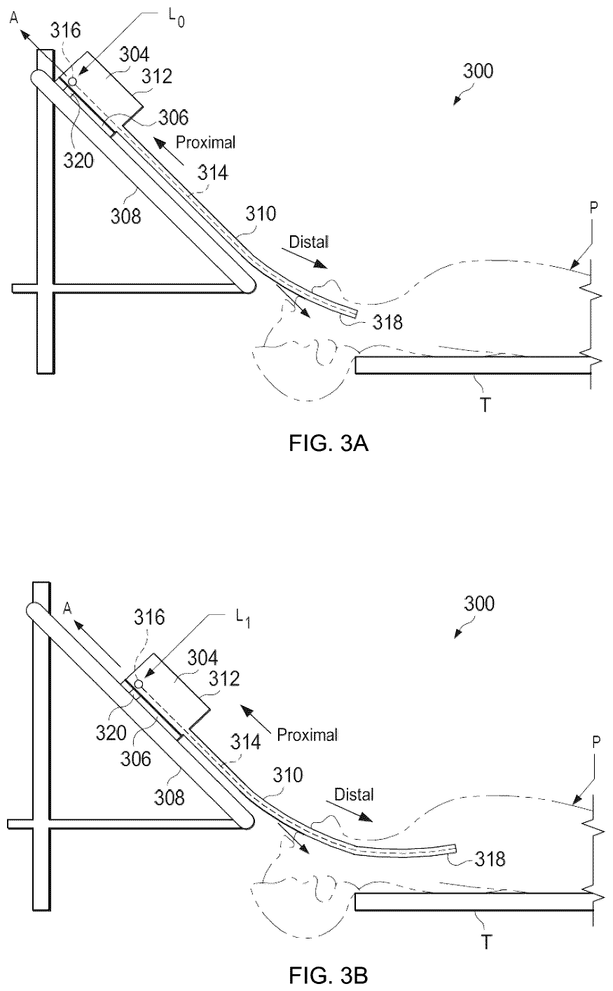

FIGS. 3A and 3B are simplified diagrams of side views of a patient coordinate space including a medical instrument mounted on an insertion assembly according to some embodiments. As shown in FIGS. 3A and 3B, a surgical environment 300 may include a patient P positioned on the patient table T. Patient P may be stationary within the surgical environment 300 in the sense that gross patient movement is limited by sedation, restraint, and/or other means. Cyclic anatomic motion, including respiration and cardiac motion, of patient P may continue. Within surgical environment 300, a medical instrument 304 is used to perform a medical procedure which may include, for example, surgery, biopsy, ablation, illumination, irrigation, suction, or electroporation. The medical instrument 304 may also be used to perform other types of procedures, such as a registration procedure to associate the position, orientation, and/or pose data captured by the sensor system 108 to a desired (e.g., anatomical or system) reference frame. The medical instrument 304 may be, for example, the medical instrument 104. In some examples, the medical instrument 304 may include an elongate device 310 (e.g., a catheter) coupled to an instrument body 312. Elongate device 310 includes one or more channels sized and shaped to receive a medical tool.

Elongate device 310 may also include one or more sensors (e.g., components of the sensor system 108). In some examples, a shape sensor 314 may be fixed at a proximal point 316 on the instrument body 312. The proximal point 316 of the shape sensor 314 may be movable with the instrument body 312, and the location of the proximal point 316 with respect to a desired reference frame may be known (e.g., via a tracking sensor or other tracking device). The shape sensor 314 may measure a shape from the proximal point 316 to another point, such as a distal end 318 of the elongate device 310. The shape sensor 314 may be aligned with the elongate device 310 (e.g., provided within an interior channel or mounted externally). In some examples, the shape sensor 314 may optical fibers used to generate shape information for the elongate device 310.

In some examples, position sensors (e.g., EM sensors) may be incorporated into the medical instrument 304. A series of position sensors may be positioned along the flexible elongate device 310 and used for shape sensing. Position sensors may be used alternatively to the shape sensor 314 or with the shape sensor 314, such as to improve the accuracy of shape sensing or to verify shape information.

Elongate device 310 may house cables, linkages, or other steering controls that extend between the instrument body 312 and the distal end 318 to controllably bend the distal end 318. In some examples, at least four cables are used to provide independent up-down steering to control a pitch of distal end 318 and left-right steering to control a yaw of distal end 318. The instrument body 312 may include drive inputs that removably couple to and receive power from drive elements, such as actuators, of a manipulator assembly.

The instrument body 312 may be coupled to an instrument carriage 306. The instrument carriage 306 may be mounted to an insertion stage 308 that is fixed within the surgical environment 300. Alternatively, the insertion stage 308 may be movable but have a known location (e.g., via a tracking sensor or other tracking device) within surgical environment 300. Instrument carriage 306 may be a component of a manipulator assembly (e.g., manipulator assembly 102) that couples to the medical instrument 304 to control insertion motion (e.g., motion along an insertion axis A) and/or motion of the distal end 318 of the elongate device 310 in multiple directions, such as yaw, pitch, and/or roll. The instrument carriage 306 or insertion stage 308 may include actuators, such as servomotors, that control motion of instrument carriage 306 along the insertion stage 308.

A sensor device 320, which may be a component of the sensor system 108, may provide information about the position of the instrument body 312 as it moves relative to the insertion stage 308 along the insertion axis A. The sensor device 320 may include one or more resolvers, encoders, potentiometers, and/or other sensors that measure the rotation and/or orientation of the actuators controlling the motion of the instrument carriage 306, thus indicating the motion of the instrument body 312. In some embodiments, the insertion stage 308 has a linear track as shown in FIGS. 3A and 3B. In some embodiments, the insertion stage 308 may have curved track or have a combination of curved and linear track sections.

FIG. 3A shows the instrument body 312 and the instrument carriage 306 in a retracted position along the insertion stage 308. In this retracted position, the proximal point 316 is at a position L0 on the insertion axis A. The location of the proximal point 316 may be set to a zero value and/or other reference value to provide a base reference (e.g., corresponding to the origin of a desired reference frame) to describe the position of the instrument carriage 306 along the insertion stage 308. In the retracted position, the distal end 318 of the elongate device 310 may be positioned just inside an entry orifice of patient P. Also in the retracted position, the data captured by the sensor device 320 may be set to a zero value and/or other reference value (e.g., I=0). In FIG. 3B, the instrument body 312 and the instrument carriage 306 have advanced along the linear track of insertion stage 308, and the distal end 318 of the elongate device 310 has advanced into patient P. In this advanced position, the proximal point 316 is at a position L1 on the insertion axis A. In some examples, the rotation and/or orientation of the actuators measured by the sensor device 320 indicating movement of the instrument carriage 306 along the insertion stage 308 and/or one or more position sensors associated with instrument carriage 306 and/or the insertion stage 308 may be used to determine the position L1 of the proximal point 316 relative to the position L0. In some examples, the position L1 may further be used as an indicator of the distance or insertion depth to which the distal end 318 of the elongate device 310 is inserted into the passageway(s) of the anatomy of patient P.

As previously noted, driving along the articulation degrees of freedom may involve articulation of the articulable body portion of the flexible elongate device, such as the distal portion of the flexible elongate device. The articulation of the articulable body portion may be driven from a proximal portion of the elongate device, e.g., using pull wires, driven by actuators. The pull wires may be actively put under mechanical tension by the actuators to cause the articulation.

The tension on the pull wires used to drive the articulation of the articulable body portion by actuators at a proximal portion of the flexible elongate device may be limited. For example, the tension may be upper-bounded to a maximum tension based on a strength of the pull wire, a maximum output (force or torque) of an actuator, software-specified limits, e.g., based on control considerations, etc. In one configuration, the tension may be dynamically upper-bounded to establish an isometrically constrained torque in the articulation space of the articulable body portion, as further discussed below.

The pull wires may further encounter friction that reduces the amount of force available to articulate the articulable body portion. The presence of friction may be non-uniform, for example, when a particular section of a pull wire is more worn than other sections of the pull wire. When such a worn section is in contact with a guiding element (e.g., a coil pipe) surrounding the pull wire, friction may be increased, in comparison to when a non-worn section is in contact with the guiding element.

The friction as described may result in an incomplete execution of a commanded or desired articulation of the articulable body portion. Specifically, a user may use an input device to provide a commanded trajectory to a commanded articulation (e.g., a specific degree of pitch and/or yaw of the articulable body portion), but the resulting actual articulation of the articulable body portion is less than the desired articulation. Such issues may be encountered particularly when a certain force/torque is needed to articulate the articulable body portion, e.g., in presence of adjacent tissue that provides resistance against the articulation. In this case, the maximum force on the pull wire(s) may not be sufficient to overcome the combination of the tissue resistance and the pull wire friction, resulting in an incomplete articulation.

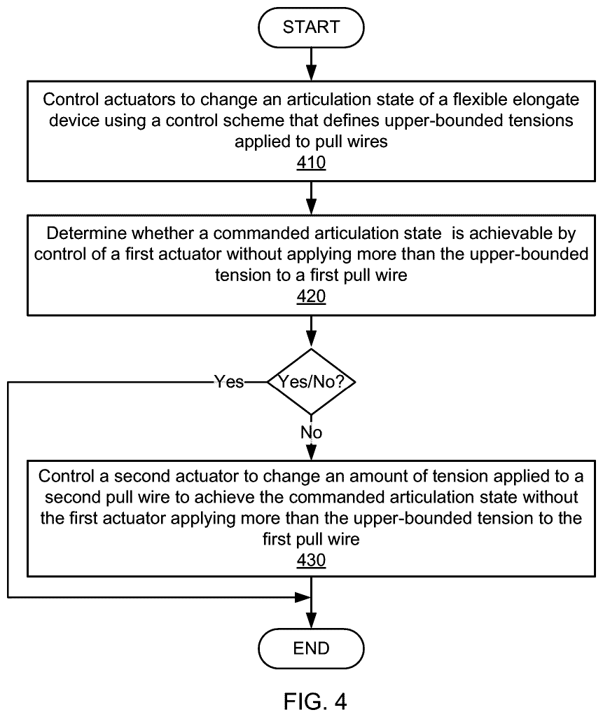

FIG. 4 shows a flowchart of methods for detecting and addressing such incomplete articulation in accordance with embodiments of the disclosure. The methods may be implemented using instructions stored on a non-transitory medium that may be executed by a computing system, e.g., the computing system 120.

While the various blocks in FIG. 4 are presented and described sequentially, some or all of the blocks may be executed in different orders, may be combined or omitted, and some or all of the blocks may be executed in parallel. Furthermore, the blocks may be performed actively or passively.

Turning to FIG. 4, in block 410, a set of actuators of the manipulator assembly are controlled to change an articulation state of a flexible elongate device in accordance with some embodiments. The flexible elongate device may be a catheter or an endoscope in a medical system as described above in FIGS. 1, 2A, 2B, 3A, and 3B and the accompanying description. In particular, a flexible elongate device may be a medical instrument that can be driven along one or more articulation degrees of freedom (e.g., about a pitch axis and/or a yaw axis) using one or more actuators. In some embodiments, a distal portion of the flexible elongate device is articulated. Where the flexible elongate device is a steerable catheter, for example, a catheter spine may form an articulable portion. Driving along the articulation degrees of freedom may be performed in order to navigate the distal end of the flexible elongate device towards a target tissue and/or to orient an end effector towards the target tissue to perform a medical operation such as a biopsy, an ablation, or an electroporation. In some embodiments, block 410 is performed in a loop in which a commanded articulation state of the flexible elongate device is obtained from a user input device, where a user may use a joystick, trackball, etc., to specify a commanded articulation state, e.g., in the form of pitch and/or yaw angles. The operations performed in block 410 may control the actuators such that an actual articulation state of the flexible elongate device follows the commanded articulation state. The trajectory taken during continuously repeated execution of block 410 results in an articulation trajectory.

One or more actuators corresponding to one or more pull wires may be used to make the flexible elongate device articulate on the articulation trajectory from an initial articulation state to a commanded articulation state. A control scheme may govern the controlling of the actuators and may include various parameters such as upper-bounded tensions (e.g., maximum tensions), lower-bounded tensions (e.g., minimum tensions), isotropic limits (e.g., established by adjustment of the upper-bounded tensions), and possibly other articulation parameters as discussed below. A minimum tension may be specified, for example, to prevent slack in the pull wires. A maximum tension may be software-specified based on, for example, the strength or durability of a pull wire. The maximum tension may also be the result of the maximum force or torque output of the corresponding actuator. Using isotropic limits, a software-specified force, torque, or tension limit may further be dynamically adjusted depending on an articulation direction to achieve an isometrically constrained articulation torque, i.e., a maximum articulation torque that is constant, regardless of the articulation direction. For example, in case of an articulation that consists of a pure pitch or yaw component, a single actuator may provide a certain torque. For articulations that include both pitch and yaw components, the actuator torque may be limited such that the total torque in the resulting direction does not exceed the torque available in a pure pitch or yaw direction. Without isotropic limits, the total torque resulting from the combination of torques applied in both pitch and yaw directions could be higher than the torque for a pure pitch or yaw component.

In block 420, a determination is made whether the commanded articulation state of the flexible elongate device is achievable in accordance with some embodiments. More specifically, it may be determined whether the commanded articulation state can be achieved by control of an actuator, without the actuator applying more than the upper-bounded tension to a pull wire controlled by the actuator. The determination may be performed based on a comparison of the commanded articulation (e.g., based on a reading obtained from an encoder at the user input device such as a trackball) and a measurement of the actual articulation of the flexible elongate device, e.g., an articulable portion. The actual articulation may be determined in various different manners, including using various types of sensors, such as a shape sensor, one or more EM sensors, etc.). A determination that the commanded articulation state is not achievable may be made when detecting that the actual articulation on the commanded trajectory from an initial articulation state to the commanded articulation state is incomplete, based on a position tracking error at the commanded articulation state. In an example of an incomplete trajectory, the tension applied by the actuator (e.g., as may be measured by an encoder) may be at a maximum tension that should result in a maximum amount of bending, but the actual articulation state has a smaller amount of bending than the maximum amount. The detection may also be performed over time, e.g., by monitoring a cumulative error of the actual articulation as it deviates from the commanded articulation. Furthermore, the detection may be performed for multiple articulation directions and actuators.

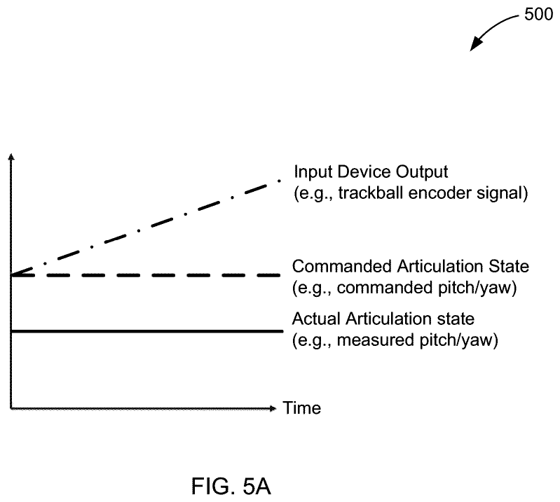

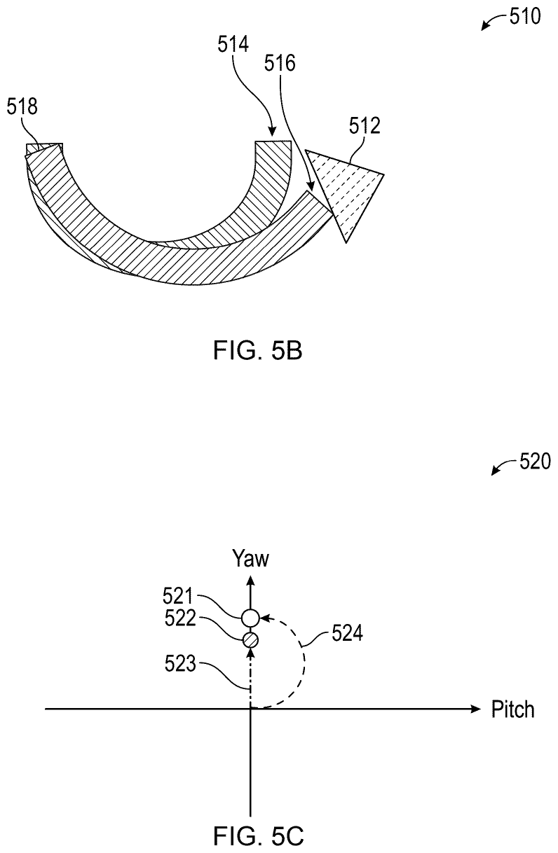

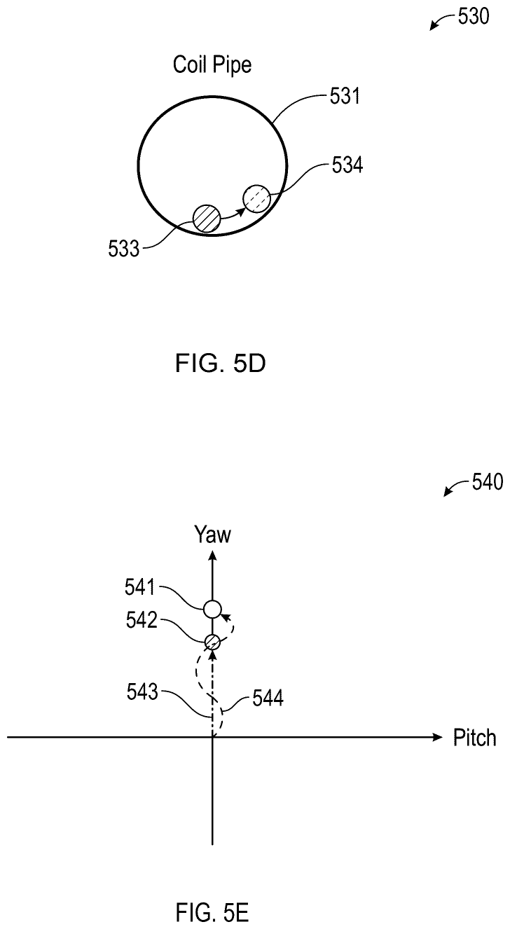

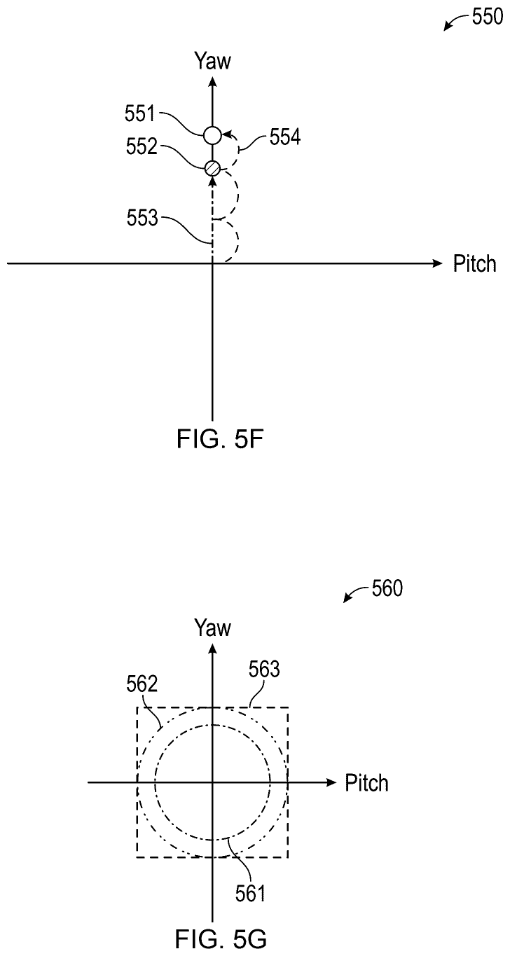

FIG. 5A illustrates a scenario in which the actual articulation state is different from the commanded articulation state. In the scenario 500, an output of the input device (e.g., a trackball encoder signal), a commanded articulation state (e.g. a commanded pitch and/or yaw), and an actual articulation state (e.g., a measured pitch and/or yaw) are shown over time. These signals may be a result of a user continuously providing an input in the same direction at the input device. This directly results in the continuously increasing input device output as shown. The input device output may be limited by a saturation function before being used to generate the commanded articulation state. In the example as shown, an upper saturation limit has already been reached, and thus the maximum tension is applied by the actuator. As a result, the commanded articulation state remains constant. In a scenario that does not suffer from the friction or external force issues as described, the actual articulation state would reach the commanded articulation state with the actuator providing maximum tension. However, in the scenario 500, the actual articulation state fails to reach the commanded articulation state despite the actuator providing maximum tension, e.g., for reasons as previously discussed. As FIG. 5A illustrates, the detection of the actual articulation state being unable to reach the commanded articulation state may be made based on the difference between the commanded articulation state and the actual articulation state, but also based on the input device output furtherer increasing with the actual articulation state failing to follow. Here, the actuator would be required to apply more than the maximum tension in order to achieve the commanded actuation state.

While the flowchart of FIG. 4 illustrates a sequential execution of blocks 410 and 420, block 420 may be executed in parallel with block 410 to provide a monitoring of the articulation state during ongoing articulations of the flexible elongate device.

When it is determined that the commanded articulation state cannot be achieved by control of an actuator without the actuator applying more than the maximum tension, or similarly for a set of actuators used to change the articulation state in block 410, the execution of the method may proceed with block 430.

As previously noted, the commanded articulation state may prove unachievable without one or more actuators exceeding maximum tension due to friction or resistance that is sufficiently high to prevent the actuator(s) controlled by the control scheme from completing the articulation to achieve the commanded articulation state. In some embodiments, a higher actuator torque than the amount corresponding with maximum tension would typically be used to complete the movement towards the commanded articulation state along a direct trajectory. However, it can be undesirable to exceed the maximum tension. As such, one or more other types of control responses may be used to achieve the commanded articulation state without any actuators applying more than the maximum tension.

FIG. 5B shows an example of an actual articulation of the flexible elongate device vs a commanded articulation of the flexible elongate device according to some embodiments. In the example 510, a tissue obstacle 512 prevents the flexible elongate device 518 from articulating sufficiently to reach the commanded articulation state 514. Instead, the flexible elongate device 518, in the example as shown, does not articulate beyond the actual articulation state 516. Accordingly, in the example, the presence of tissue obstacle 512 renders the commanded trajectory (towards the commanded articulation state 514) unachievable. The tissue obstacle 512 may, for example, be in contact with the flexible elongate device 518, such that friction between the tissue obstacle 512 and the flexible elongate device 518 prevents further articulation. Alternatively, internal friction along the pull wire(s) may also result in a failure to reach the commanded articulation state 514 on the commanded trajectory. Increased internal friction may occur, for example with increasing wear of the flexible elongate device over time. For example, the pull wires and/or pull-wire supporting structures may be coated with friction reducing material (e.g., Teflon), which may wear off eventually. The resulting increase in friction reduces the amount of force or torque available for articulation of the flexible elongate device. The resulting friction may be non-uniform and may depend on a degree and direction of the articulation as a result of particular sections of a pull wire being more worn than other sections of the same pull wire. When such a worn section is in contact with a guiding element (e.g., a coil pipe) surrounding the pull wire, friction may increase in comparison to contact with an unworn section. An incomplete articulation may also be a result of internal components of the flexible elongate device providing resistance to the articulation. For example, insertion of an instrument into a lumen of the flexible elongate device may increase the overall stiffness of the flexible elongate device. If such a component is put under tension for certain articulation directions only, the internal resistance may be more in some articulation directions than in others.

In block 430, the control of the flexible elongate device is updated such that the commanded articulation state can be reached. In some embodiments, the amount of tension applied to one or more pull wires is changed without exceeding maximum tension. For example, if it was determined that the commanded articulation cannot be achieved by control of a first actuator without the first actuator applying more tension than the upper-bounded tension to a first pull wire, in block 430, a second actuator may be controlled to change an amount of tension applied to a second pull wire to achieve the commanded articulation state without the first actuator applying more than the upper-bounded tension to the first pull wire. The second pull wire may be responsible for articulation along a different axis from the first pull wire. As a result of this change, the flexible elongate device may take a different articulation trajectory to the commanded articulation state, thereby potentially avoiding the reason for initially failing to reach the commanded articulation state. The articulation in a different direction may have different implications that may help enabling reaching the commanded articulation state.

For example, FIGS. 5C and 5D are illustrations of an adjustment of a commanded direct articulation trajectory to a non-direct articulation trajectory according to some embodiments. The non-direct articulation trajectory may be used when the direct articulation trajectory fails, such as may be caused by friction between pull wires and other structural features of the flexible elongate device around or near the pull wires. A non-direct articulation trajectory between a first (e.g., initial) articulation state and a second (e.g., commanded) articulation state refers to an articulation trajectory that includes movements in opposite directions along one or more articulation axes (e.g., pitch or yaw). The non-direct articulation trajectory includes offsetting movements, or multiple offsetting movements, that facilitate the flexible elongate device's ability to reach the second articulation state. In contrast, a direct articulation trajectory between a first and second articulation state refers to an articulation trajectory that includes only movements in a single direction along any of the articulation axes (e.g., pitch, yaw, or any other direction, e.g., combinations of pitch and yaw). Here, there are no offsetting movements in any articulation axis, which provides for efficient movement when there is no struggling or incomplete motion.

In FIG. 5C, a user input specifies an articulation to a commanded articulation state 521. The articulation follows a direct articulation trajectory 523 (entirely in a yaw direction, in the example), but terminates at the initial articulation state 522, thereby failing to reach the commanded articulation state 521. When the initial articulation state 522 on the commanded trajectory is detected as incomplete, a non-direct articulation trajectory 524 is determined. The non-direct articulation trajectory involves an articulation component in the pitch direction. Accordingly, while the direct articulation trajectory may have been driven by a single actuator (or two actuators in an antagonistic driving scheme) for the yaw direction, the non-direct articulation trajectory further involves a second actuator (or two actuators in an antagonistic driving scheme) for the pitch component. Assuming a pull wire for pitch and a pull wire for yaw (or, in an antagonistic control scheme, a pair of pull wires for pitch and a pair of pull wires for yaw), the non-direct articulation trajectory 524 may be a result of operating the actuator(s) associated with the pitch direction to increase and decrease tension on the corresponding pull wires. In the example as shown, first, a pitch component is superimposed in a direction away from the yaw axis, followed by pitch component towards the yaw axis. More generally, the superimposed component may be in a direction orthogonal to the direct articulation trajectory. The superimposition of a pitch component may be performed while the actuator(s) associated with the yaw direction is continuously commanded to hold the commanded articulation state 521. During the superimposition, the tension on the pull wire(s) associated with the articulation towards the commanded articulation state may be at an upper-bounded or maximum tension. As the component in the pitch direction is superimposed as illustrated, the actual articulation may gradually proceed from the initial articulation state 522 to the commanded articulation state 521. The resulting altered trajectory directionally deviates from the commanded trajectory. A first segment diverges from the commanded trajectory with a pitch component away from the commanded trajectory, and a second segment converges to the commanded trajectory with a pitch component towards the commanded trajectory and terminating at the commanded articulation state.

More generally, a direct articulation trajectory may include pitch and/or yaw components in a single direction that do not offset each other. Accordingly, the resulting trajectory may be a substantially straight trajectory (e.g., with only minor deviations from a straight path, resulting from manual user input) in any direction in the articulation space. In contrast, a non-direct articulation trajectory may involve a pitch component moving in opposite, offsetting directions or a yaw component moving in opposite, offsetting directions, thereby achieving the commanded articulation using a non-direct articulation trajectory.

The altered trajectory reaches the commanded articulation state that is unreachable on the commanded trajectory for various possible reasons:
As previously described, articulation of the flexible elongate device may be driven by the application of tensions to pull wires. In a yaw-pitch configuration as illustrated in FIG. 5C, there may be four pull wires (one pair for antagonistic control of pitch, and one pair for antagonistic control of yaw). Each pull wire may encounter friction, and the friction may be non-uniform, with some sections of the pull wires or coil pipes for the pull wires being more worn than others. This results in the friction being dependent on degree and direction of the articulation. Accordingly, with the altered trajectory being different from the original trajectory, the friction that is encountered may be less. An example 530 is provided in FIG. 5D, which shows a coil pipe for a pull wire. The pull wire is shown in an original position 533 which may correspond to a worn zone with increased friction of the coil pipe 531. The pull wire may be in the original position 533 for execution of the commanded trajectory 523. The pull wire is further shown in an altered position 534 in which the pull wire may be, at least temporarily, during the execution of the alternate trajectory 524.
In the example as shown in FIG. 5C, the original trajectory is purely in a yaw direction. A single actuator may be activated to drive this movement in the yaw direction. This single actuator may be limited in its force or torque output, as previously described. In the example of FIG. 5C, the output of the actuator is not sufficient to reach the commanded articulation state, e.g., because of friction. The altered trajectory involves movement in pitch and yaw direction. Accordingly, two actuators are involved. The two actuators generate an output that is higher than the output produced by the single actuator, thereby facilitating reaching the commanded articulation state.
In case of tissue contact with the flexible elongate device preventing the completion of the original trajectory, the altered trajectory being in a different direction may result in less friction or resistance by the tissue.

Continuing with the discussion of block 430 of the method shown in FIG. 4, the controlling of the one or more actuators to change the articulation state of the flexible elongate device may be performed in different manners. As previously described, the amount of tension applied to one or more tension members is changed to make the flexible elongate device articulate in a different direction, thereby potentially avoiding the reason for initially failing to reach the commanded articulation state. In another example, the tensions are altered to generate non-direct articulation trajectories as shown in FIGS. 5E and 5F.

In the example 540 shown in FIG. 5E, after determining that the commanded articulation state 541 cannot be reached on the direct articulation trajectory 543 with the articulation failing to proceed beyond the initial articulation state 542, a non-direct articulation trajectory 544 is chosen to reach the commanded articulation state 541. The non-direct articulation trajectory 544 includes repetitions of segments that alternatingly diverge and converge from, to the commanded trajectory, in alternating directions. Assuming antagonistic pairs of pull wires (one pair for pitch, one pair for yaw), the non-direct articulation trajectory 544 may be a result of operating the actuators associated with the pitch direction in an alternating manner. This may be performed while the actuators associated with the yaw direction are continuously commanded to hold the commanded articulation state 541. As the component in the pitch direction is superimposed as illustrated, the actual articulation may gradually proceed from the initial articulation state 542 to the commanded articulation state 541.

In the example 550 shown in FIG. 5F, after determining that the commanded articulation state 551 cannot be reached on the direct articulation trajectory 553 with the articulation failing to proceed beyond the initial articulation state 552, a non-direct articulation trajectory 554 is chosen to reach the commanded articulation state 551. The non-direct articulation trajectory 554 includes repetitions of segments that alternatingly diverge and converge from, to the commanded trajectory, in the same direction. Assuming a pull wire for pitch and a pull wire for yaw (or, in an antagonistic control scheme, a pair of pull wires for pitch and a pair of pull wires for yaw), the non-direct articulation trajectory 554 may be a result of operating the actuator(s) associated with the pitch direction as previously discussed in reference to FIG. 5C. This may be performed while the actuator(s) associated with the yaw direction is continuously commanded to hold the commanded articulation state 551. As the component in the pitch direction is superimposed as illustrated, the actual articulation may gradually proceed from the initial articulation state 552 to the commanded articulation state 551.

The altered trajectories of FIGS. 5E and 5F reach the commanded articulation states for possible reasons as previously described in reference to FIGS. 5C and 5D.

In the examples 540 and 550, the execution of the repeated segments may be coordinated with the user input at the user input device. Assume, for example, that the user input device to provide the commanded trajectory to the commanded articulation is a trackball. In such a configuration, the user may input the commanded trajectory in increments, e.g., based on the intrinsic range-of-motion limitation of a trackball requiring the user to repeatedly reposition the fingers on the trackball. In this case, each of the repeated segments may be executed as the user operates the trackball to complete one increment of the commanded trajectory.

As shown in FIGS. 5C-5F, the non-direct articulation trajectory may include changes in a direction component at different frequencies. While in FIG. 5C, a lower frequency change is shown, FIGS. 5E and 5F illustrate higher frequency changes. Additional variations may be applied. For example, assuming that there is a first directional component along one axis (e.g., the yaw axis) and a second directional component along the pitch axis, the contributions in these two directions may be varied such that either the first articulation component dominates over the second articulation component in one articulation direction, and the second articulation component dominates over the first articulation component in another articulation direction.

While an antagonistic driving scheme is not required for the behaviors illustrated in FIGS. 5B-5F, embodiments of the disclosure are compatible with such implementations. In one example, two pull wires, on opposite sides, may control the articulation along a single axis. In one embodiment, there are four pull wires total, with pairs of pull wires controlling orthogonal pitch and yaw axes of an articulable portion of a flexible elongate device. In such an implementation, antagonistically-operating actuators may be used to both drive articulation as well as set or maintain a minimum required tension on the pull wires. When incomplete execution of a commanded articulation is detected, the minimum required tension may be lowered, thereby increasing the amount of torque available for the articulation.

Further, while in the illustrations of FIGS. 5C-5F the direct articulation trajectory is shown in alignment with the yaw axis, embodiments of the disclosure generalize to direct articulation trajectories in any direction.

Continuing with the discussion of block 430 of the method shown in FIG. 4, the commanded articulation state may also be reached without directionally deviating from the direct articulation trajectory. This is discussed in reference to FIG. 5G.

FIG. 5G shows the modulation of actuator torques or forces to reach the commanded articulation state. A scenario 560 involving an antagonistic driving along pitch (two actuators) and yaw (two actuators) is shown. More generally, the antagonistic driving may be performed by pairs of actuators along any common articulation axis. An isometrically constrained torque is visualized, with the torque limit being the same, regardless of the direction. Accordingly, the torques are limited to a circular region in the pitch/yaw plane 561, 562. The torque limit 561 is a result of a minimum required tension on the pull wires. In presence of the minimum required tension in an antagonistic driving scheme, the overall torque available for articulation is reduced by an opposing torque associated with the minimum required tension on the opposing pull wire. Minimum tensions may be imposed for various reasons, e.g., to prevent slack in the pull wires. The torque limit 562 is a result of decreasing the minimum tension without increasing the upper-bounded tension, while still requiring an isometric torque limit. For example, the minimum tension is reduced to zero, thereby entirely eliminating the torque produced on the opposing pull wire. Reducing the minimum tension along a common articulation axis alone may allow completion of the articulation on the direct articulation trajectory. Reducing the minimum tension may also facilitate the completion of the articulation on the non-direct articulation trajectory. Alternatively, the higher torque limit 562 in comparison to the torque limit 561 may also be a result of increasing the upper-bounded tension (maximum tension) on the pull wires. As an additional or alternative step, the isometric torque requirement may be disabled, as illustrated by the torque limit 563. The disabling of the isometric torque constraint may enable each of the actuators to apply a tension up to the maximum tension on each of the pull wires, regardless of the articulation direction in the pitch-yaw space. While the elimination of the isometric torque requirement does not result in a further increase of the torque in the directions along the pitch and yaw axes, for any off-axis directions, the torque is increased, as a result of two actuators jointly contributing to the torque, without exceeding the maximum tension. A maximum torque may be realized at the 45° angles, where two actuators (one yaw actuator and one pitch actuator) contribute equally.

A non-direct articulation trajectory consisting of segments oriented such that they involve equal contribution of pitch and yaw components may, thus, be the trajectory towards the target articulation state that can be completed with a maximum torque.

Continuing with the discussion of block 430, a number of different approaches to changing the articulation state have been described. When entering block 430, one or more of the described operations may be automatically selected, e.g., after determining that the initial articulation state on the commanded trajectory by the flexible elongate device is incomplete based on a position tracking error at the commanded articulation state. The method may execute one or more of the operations to identify a trajectory that allows the commanded articulation state, provided by the user at the user input device, to be reached. Alternatively, the user may manually activate one or more of these operations. The user may then provide input to proceed on the commanded articulation trajectory, while the flexible elongate device is controlled with the described trajectory and/or force/torque/tension modifications superimposed until the commanded articulation state is reached. These operations, thus, support the user, by providing an assistance without requiring the user to alter the input they provide to control articulation. While the input has been described as user-provided (e.g., a user providing the input at an input device), the input may also be autonomously or semi-autonomously provided by an algorithm.

One or more components of the embodiments discussed in this disclosure, such as control system 112, may be implemented in software for execution on one or more processors of a computer system. The software may include code that when executed by the one or more processors, configures the one or more processors to perform various functionalities as discussed herein. The code may be stored in a non-transitory computer readable storage medium (e.g., a memory, magnetic storage, optical storage, solid-state storage, etc.). The computer readable storage medium may be part of a computer readable storage device, such as an electronic circuit, a semiconductor device, a semiconductor memory device, a read only memory (ROM), a flash memory, an erasable programmable read only memory (EPROM); a floppy diskette, a CD-ROM, an optical disk, a hard disk, or other storage device. The code may be downloaded via computer networks such as the Internet, Intranet, etc. for storage on the computer readable storage medium. The code may be executed by any of a wide variety of centralized or distributed data processing architectures. The programmed instructions of the code may be implemented as a number of separate programs or subroutines, or they may be integrated into a number of other aspects of the systems described herein. The components of the computing systems discussed herein may be connected using wired and/or wireless connections. In some examples, the wireless connections may use wireless communication protocols such as Bluetooth, near-field communication (NFC), Infrared Data Association (IrDA), home radio frequency (HomeRF), IEEE 802.11, Digital Enhanced Cordless Telecommunications (DECT), and wireless medical telemetry service (WMTS).

Various general-purpose computer systems may be used to perform one or more processes, methods, or functionalities described herein. Additionally or alternatively, various specialized computer systems may be used to perform one or more processes, methods, or functionalities described herein. In addition, a variety of programming languages may be used to implement one or more of the processes, methods, or functionalities described herein.

While certain embodiments and examples have been described above and shown in the accompanying drawings, it is to be understood that such embodiments and examples are merely illustrative and are not limited to the specific constructions and arrangements shown and described, since various other alternatives, modifications, and equivalents will be appreciated by those with ordinary skill in the art.

What is claimed is:

1. A medical system, comprising:
a manipulator assembly comprising a plurality of actuators configured to control an articulation state of a flexible elongate device based on changing a plurality of tensions applied to a plurality of pull wires of the flexible elongate device; and
a control system coupled to the manipulator assembly, the control system configured to:
control the plurality of actuators to change the articulation state of the flexible elongate device using a control scheme, the control scheme defining an upper-bounded tension applied to each of the plurality of pull wires;
determine that a commanded articulation state of the flexible elongate device cannot be achieved by control of a first actuator of the plurality of actuators without the first actuator applying more than the upper-bounded tension to a first pull wire of the plurality of pull wires; and
control a second actuator of the plurality of actuators to change an amount of tension applied to a second pull wire of the plurality of pull wires to achieve the commanded articulation state without the first actuator applying more than the upper-bounded tension to the first pull wire.

2. The medical system of claim 1, wherein determining that the commanded articulation state cannot be achieved by control of the first actuator comprises determining that an actual articulation on a commanded trajectory from an initial articulation state to the commanded articulation state by the flexible elongate device is incomplete.

3. The medical system of claim 2,
wherein determining that the actual articulation on the commanded trajectory by the flexible elongate device is incomplete comprises determining a position tracking error at the commanded articulation state.

4. The medical system of claim 1, wherein:
the first and second pull wires control different articulation axes;
the commanded articulation state cannot be achieved by control of the first actuator when using a direct articulation trajectory from an initial articulation state to the commanded articulation state, and
the second actuator is controlled to achieve the commanded articulation state using a non-direct articulation trajectory from the initial articulation state to the commanded articulation state.

5. The medical system of claim 4,
wherein the non-direct articulation trajectory comprises a first segment that diverges from a direct articulation trajectory from the initial articulation state to the commanded articulation state and a second segment that converges to the direct articulation trajectory.

6. The medical system of claim 5, wherein the second segment converges with the direct articulation trajectory at the commanded articulation state.

7. The medical system of claim 5, wherein the non-direct articulation trajectory comprises a repetition of the first segment and the second segment.

8. The medical system of claim 5,
wherein the non-direct articulation trajectory comprises a first directional component in a first articulation direction that dominates over a second directional component in a second articulation direction, and
wherein in the first segment and the second segment of the non-direct articulation trajectory, the second directional component is increased over the first directional component of the commanded trajectory.

9. The medical system of claim 1, wherein the upper-bounded tension is a maximum tension.

10. The medical system of claim 1,
wherein the control scheme defines a minimum tension applied to each of the plurality of pull wires;
the first pull wire and the second pull wire control a common articulation axis; and
the control system is configured to control the second actuator to decrease the tension applied to the second pull wire to below the minimum tension to achieve the commanded articulation state.

11. The medical system of claim 10,
wherein the minimum tension is decreased to zero.

12. The medical system of claim 1,
wherein the control scheme dynamically sets the upper-bounded tension applied to each of the plurality of pull wires to isometrically constrain torque in an articulation space formed by combinations of a first articulation direction and a second articulation direction of the flexible elongate device.

13. The medical system of claim 12, wherein the control system is further configured to:
disable the isometric constraining of the torque when controlling the second actuator of the plurality of actuators to change the amount of tension applied to the second pull wire of the plurality of pull wires to achieve the commanded articulation state without the first actuator applying more than the upper-bounded tension to the first pull wire.

14. The medical system of claim 13, wherein when disabling the isometric constraining of the torque, the upper-bounded tension is a maximum tension.

15. The medical system of claim 13, wherein the second actuator is controlled to achieve the commanded articulation state using a non-direct articulation trajectory from an initial articulation state to the commanded articulation state.

16. A non-transitory machine-readable medium comprising a plurality of machine-readable instructions executed by one or more processors associated with a medical system,
the medical system comprising a manipulator assembly comprising a plurality of actuators configured to control an articulation state of a flexible elongate device based on changing a plurality of tensions applied to a plurality of pull wires of the flexible elongate device,
the plurality of machine-readable instructions causing the one or more processors to perform a method comprising:
controlling the plurality of actuators to change the articulation state of the flexible elongate device using a control scheme, the control scheme defining an upper-bounded tension applied to each of the plurality of pull wires;
determining that a commanded articulation state of the flexible elongate device cannot be achieved by control of a first actuator of the plurality of actuators without the first actuator applying more than the upper-bounded tension to a first pull wire of the plurality of pull wires; and
controlling a second actuator of the plurality of actuators to change an amount of tension applied to a second pull wire of the plurality of pull wires to achieve the commanded articulation state without the first actuator applying more than the upper-bounded tension to the first pull wire.

17. The non-transitory machine-readable medium of claim 16, wherein determining that the commanded articulation state cannot be achieved by control of the first actuator comprises determining that an actual articulation on a commanded trajectory from an initial articulation state to the commanded articulation state by the flexible elongate device is incomplete.

18. The non-transitory machine-readable medium of claim 17,
wherein determining that the actual articulation on the commanded trajectory by the flexible elongate device is incomplete comprises determining a position tracking error at the commanded articulation state.

19. The non-transitory machine-readable medium of claim 16, wherein:
the first and second pull wires control different articulation axes,
the commanded articulation state cannot be achieved by control of the first actuator when using a direct articulation trajectory from an initial articulation state to the commanded articulation state, and
the second actuator is controlled to achieve the commanded articulation state using a non-direct articulation trajectory from the initial articulation state to the commanded articulation state.

20. A method for operating a medical system,
the medical system comprising a manipulator assembly comprising a plurality of actuators configured to control an articulation state of a flexible elongate device based on changing a plurality of tensions applied to a plurality of pull wires of the flexible elongate device,
the method comprising:
controlling the plurality of actuators to change the articulation state of the flexible elongate device using a control scheme, the control scheme defining an upper-bounded tension applied to each of the plurality of pull wires;
determining that a commanded articulation state of the flexible elongate device cannot be achieved by control of a first actuator of the plurality of actuators without the first actuator applying more than the upper-bounded tension to a first pull wire of the plurality of pull wires; and
controlling a second actuator of the plurality of actuators to change an amount of tension applied to a second pull wire of the plurality of pull wires to achieve the commanded articulation state without the first actuator applying more than the upper-bounded tension to the first pull wire.
